(19) United States
(12) Patent Application Publication
(10) Pub. No.: US 2007/0191930 A1
(43) Pub. Date: Aug. 16, 2007

(54) Endovascular graft adapter

(76) Inventors: Paul Lucas, Ellicott City, MD (US); Elliott Badder, Lutherville, MD (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/354,039

(22) Filed: Feb. 15, 2006

(51) Int. Cl. A61F 2/06

(52) U.S. Cl. 623/1.36

(57) ABSTRACT

An endovascular graft adapter for use in abdominal aortic aneurysms comprising a graft adapter body defining an upper graft adapter section member and a lower graft adapter section member. The upper and lower graft adapter section members respectively have a pair of cut-out sections formed through sidewalls thereof and both pairs of cut-out sections are respectively aligned with the right and left renal arteries. The upper graft adapter section member has an upper axially directed through passage extending from a proximal end to a distal end thereof and the lower graft adapter section member has a lower axially directed through passage extending from a proximal end to a distal end thereof. The upper and lower axially directed through passages are in alignment each with respect to the other.

### BACKGROUND OF THE INVENTION

1. Field of the Invention

The present invention is directed to an endovascular graft adapter for use in abdominal aortic aneurysms. The present endovascular graft adapter is adapted to be used with conventional endovascular stent grafts to benefit patients that are not proper candidates for the use of conventional endovascular stent grafts.

2. Description of the Prior Art

Each year, physicians diagnose approximately 200,000 people in the United States with abdominal aortic aneurysms. Of those diagnosed, many have abdominal aortic aneurysms that are threatening enough to cause death from a rupture of the aneurysm if left untreated.

There are many stents and endovascular stent grafts currently available for use in abdominal aortic aneurysms. However, such endovascular stent grafts are limited based on the particular anatomy of a patient. Specifically, one such limitation is based on the size of the abdominal aorta at the level of the neck or the region just distal to the renal arteries. In many instances of abdominal aortic aneurysms, the aneurysm occurs just below or distal to the renal arteries. Thus, conventional endovascular stent grafts can not be used in these cases because there is not enough aortic wall necessary to support such a stent graft. In other instances, since conventional stent grafts are available only in particular sizes, if the size of the aorta is not appropriate, a conventional stent graft can not be used.

Therefore, many patients do not qualify based on the anatomy of their aorta or the actual location of the aneurysm for conventionally known endovascular stent grafts.

Furthermore, conventional endovascular stent grafts do not provide for placement above the renal arteries while accommodating more official portions of the mesenteric or renal vessels, i.e. the superior mesenteric artery and the right and left renal arteries.

### SUMMARY OF THE INVENTION

The present invention provides the means for treating abdominal aortic aneurysms with conventionally known standard endovascular stent graft systems. The invention is an endovascular graft adapter that is adapted to be coupled with a secondary, conventionally known endovascular stent graft for broader treatment options.

It is one object of the present invention to provide an endovascular graft adapter for use in abdominal aortic aneurysms comprising a graft adapter body defining an upper graft adapter section member and a lower graft adapter section member. The upper graft adapter section member has a pair of upper graft adapter section member cut-out sections formed through a sidewall thereof and respectively aligned with the right and left renal arteries. The lower graft adapter section member has a pair of lower graft adapter section member cut-out sections formed through a sidewall thereof and respectively aligned with the right and left renal arteries. The upper graft adapter section member has an upper axially directed through passage extending from a proximal end to a distal end thereof and the lower graft adapter section member has a lower axially directed through passage extending from a proximal end to a distal end thereof. The upper and lower axially directed through passages are in alignment each with respect to the other.

It is another object of the present invention to provide an endovascular graft adapter that is adapted to be used in combination with conventional endovascular stent grafts to treat patients in which conventional endovascular stent grafts are not appropriate due to poor support for the stent graft.

It is yet another object of the present invention to provide an endovascular graft adapter that is adapted to be used in combination with conventional endovascular stent grafts to treat patients in which conventional endovascular stent grafts are not appropriate due to the size of the aorta.

It is a further object of the present invention to provide an endovascular graft adapter that provides for supra renal fixation while accommodating more official portions of the mesenteric or renal vessels, i.e. the superior mesenteric artery and the right and left renal arteries.

### BRIEF DESCRIPTION OF THE DRAWINGS

FIG. 1 is a perspective blow-out view of the endovascular graft adapter showing the upper graft adapter section member and the lower graft adapter section member;

FIG. 2 is an illustration of the endovascular graft adapter supra renally fixated and a cross-sectional view of the abdominal aorta;

FIG. 3 is a front perspective blow-out view of an alternate embodiment of a one piece endovascular graft adapter;

FIG. 4 is an illustration of the alternate embodiment of the endovascular graft adapter supra renally fixated and showing a cross-sectional view of the abdominal aorta;

FIG. 5 is an illustration of the endovascular graft adapter supra renally fixated with a metallic stent section at a proximal end thereof and a cross-sectional view of the abdominal aorta with an abdominal aortic aneurysm;

FIG. 6 is a front perspective blow-out view of the endovascular graft adapter with a metallic stent-section shown at a proximal end thereof;

FIG. 7 is an illustration of the alternate embodiment of the endovascular graft adapter supra renally fixated with a metallic stent section at a proximal end thereof and a cross-sectional view of the abdominal aorta with an abdominal aortic aneurysm;

FIG. 8 is a front perspective blow-out view of the alternate embodiment of endovascular graft adapter with a metallic stent section at a proximal end thereof;

FIG. 9 is an illustration of the endovascular graft adapter supra renally fixated with a plurality of radiopaque markers thereon and a cross-sectional view of the abdominal aorta showing blood flow through the endovascular graft adapter;

FIG. 10 is a front perspective blow-out view of the endovascular graft adapter with a plurality of radiopaque markers thereon.

### DETAILED DESCRIPTION OF THE PREFERRED EMBODIMENTS

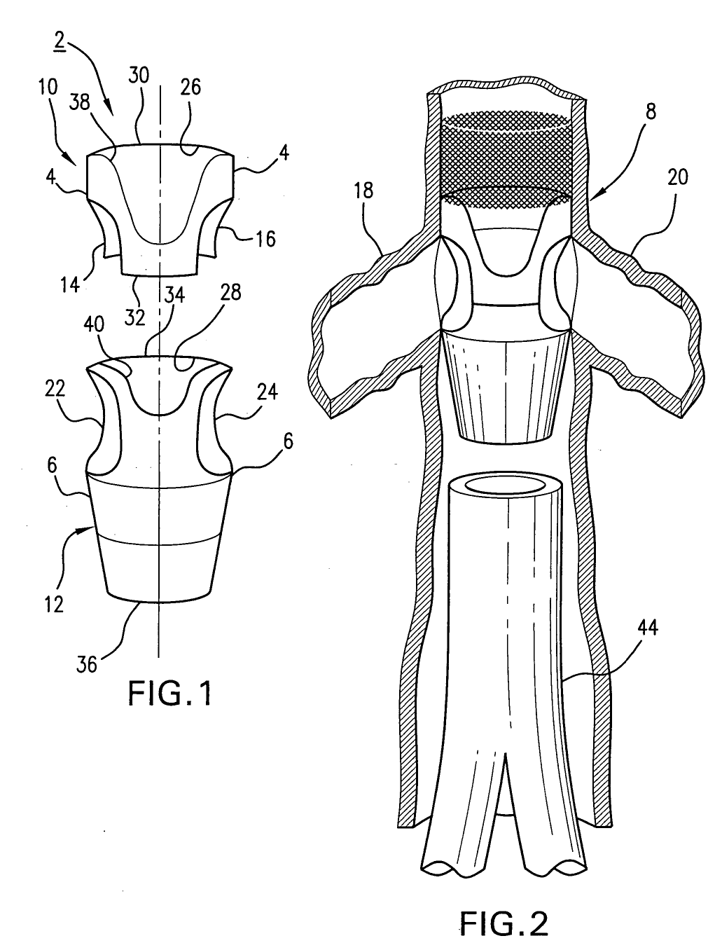

As shown in FIGS. 1-2, 5-6 and 9-10, an endovascular graft adapter 2 that is used in abdominal aortic aneurysms. The endovascular graft adapter 2, in a preferred embodiment, is formed of a two-piece structure. The endovascular graft adapter 2 is placed within the aorta 8 above the renal arteries 18, 20. A secondary stent graft 44 well-known in the art is attached to a distal end 36 of the endovascular graft adapter 2. In this manner, patients who are not eligible for a conventional endovascular stent graft 44 due to poor anatomy (conventional endovascular stent graft 44 cannot fit or be placed properly in the aorta) can be treated with the use of endovascular graft adapter 2.

The endovascular graft adapter 2 of the present invention is located above the renal arteries 18, 20. Such placement is accomplished without impeding blood flow to the renal arteries 18, 20 or the superior mesenteric artery which allows fluid communication through the optimally positioned cutouts 14, 16, 22 and 24. This type of supra renal fixation is accomplished as previously described, with the ability to accommodate more official portions of the mesenteric or renal vessels.

As seen in FIGS. 1, 6, and 10, the endovascular graft adapter 2 is comprised in two-piece formation which defines an upper graft adapter section member 10 and a lower graft adapter section member 12. The upper graft adapter section member 10 has a pair of upper graft adapter section member cut-out sections 14, 16. The upper graft adapter section member cut-out sections 14, 16 are formed through a sidewall 4 of the upper graft adapter section member 10. As shown in FIGS. 2, 5, and 9, the upper graft adapter section member 10 is positioned internal the aorta 8 and the upper graft adapter section member cut-out sections 14, 16 are respectively aligned with the right and left renal arteries 18, 20. This manner of alignment maintains blood flow through the right and left renal arteries 18, 20 when the upper graft adapter section member 10 is positioned in the aorta 8. Furthermore, the endovascular graft adapter 2 uses portions of the aortic wall above the renal arteries for stability and fixation by virtue of the upper graft adapter section member cut-out sections 14, 16 which maintain blood flow to the renal arteries 18, 20.

Further referring to FIGS. 1, 6, and 10, the lower graft adapter section member 12 has a pair of lower graft adapter section member cut-out sections 22, 24. The lower graft adapter section member cut-out sections 22, 24 are formed through a sidewall 6 of the lower graft adapter section member 12. As shown in FIGS. 2, 5, and 9, when the lower graft adapter section member 12 is positioned internal the aorta 8, the lower graft adapter section member cut-out sections 22, 24 are respectively aligned with the right and left renal arteries 18, 20. This manner of alignment maintains blood flow through the right and left renal arteries 18, 20 when the lower graft adapter section member 12 is placed in the aorta.

The combination of the cut-out sections 14, 16, 22, 24 of the respective upper graft adapter section member 10 and the lower graft adapter section member 12 allow for supra renal fixation of the endovascular graft adapter 2 in patients that are not appropriate candidates for an endovascular approach while allowing for a proper flow of blood through the endovascular graft adapter 2 to the right and left renal arteries 18, 20.

The upper graft adapter section member cut-out sections 14, 16 are located on sidewalls of the upper graft adapter section members 10 to accommodate varying orientations of the right and left renal arteries 18, 20 depending on the anatomy of a particular patient. Similarly, the lower graft adapter section member cut-out sections 22, 24 can be oriented in a manner on the sidewalls of the lower graft adapter section members 12 depending on the anatomy of the patient.

As seen in FIGS. 1, 6, and 10, the upper graft adapter section member 10 has an upper axially directed through passage 26 that extends from a proximal end 30 to a distal end 32 thereof. This through passage 26 allows blood to flow from the aorta 8 through the upper graft adapter section member 10 from the proximal end 30 towards the distal end 32 thereof. Aligning with the upper axially directed through passage 26 is a lower axially directed through passage 28 defined by the lower graft adapter section member 12. The lower axially directed through passage 26 extends from a proximal end 34 of the lower graft adapter section member 12 to a distal end 36 thereof. As detailed above, the upper and lower axially directed through passages 26, 28 are in alignment each with respect to the other. The lower axially directed through passage 28 allows blood flow from the upper axially directed through passage 26 through the lower graft adapter section 12 from the proximal end 34 to the distal end 36 thereof.

The combination of the upper axially directed through passage 26 and the lower axially directed through passage 28 form an endovascular graft adapter through passage 26, 28 that extends from a proximal end of the endovascular graft adapter 2 to a distal end thereof. The endovascular graft adapter through passage 26, 28 allows blood flow, without restriction, from the aorta 8 through the endovascular graft adapter 2 from a proximal to a distal end thereof.

Referring to FIGS. 2, 5, and 9, the endovascular graft adapter 2 is shown placed within the aorta 8 with the upper graft adapter section member 10 and the lower graft adapter section member 12 secured each to the other. Specifically, the proximal end 34 of the lower graft adapter section member 12 is coupled to the distal end 32 of the upper graft adapter section member 10. Once the upper graft adapter section member 10 and the lower graft adapter section member 12 are secured each to the other, the upper graft adapter section member cut-out sections 14, 16 and the lower graft adapter section member cut-out sections 22, 24 are in alignment with each other thereby defining endovascular graft adapter cut-out sections that are respectively aligned with the right and left renal arteries 18, 20. The combination of the lower graft adapter section member 12 and the upper graft adapter section member 10 form the endovascular graft adapter 2 and the endovascular graft adapter 2 is formed in such a manner which allows for supra renal fixation thereof without inhibiting blood flow to the right and left renal arteries 18, 20.

The endovascular graft adapter 2 uses blood flow radial forces through the upper and lower graft adapter section member 10, 12 to maintain a coupling effect. The radially outward force of blood flow along with the self-expanding nature of the endovascular graft adapter 2 (detailed in following paragraphs) further ensures the overall integrity thereof.

The endovascular graft adapter 2 is self-expanding and thus pushes out circumferentially to seal the blood vessel in which it is placed, i.e. the aorta 8. As previously described, the self-expanding nature of the overall endovascular graft adapter 2 is one method by which the lower graft adapter section member 12 and the upper graft adapter section member 10 remain coupled each to the other.

Referring to FIGS. 1, 6, and 10, the upper graft adapter section member 10 further comprises an upper graft adapter section member third cut-out section 38. The upper graft adapter section member third cut-out section 38 is formed on the upper graft adapter section member 10 to correspond with the superior mesenteric artery. In most cases, the upper graft adapter section member third cut-out section 38 will be formed on an anterior wall of the upper graft adapter section member 10 but can be varied based on the anatomy of a patient. Furthermore, in most cases, the upper graft adapter section member third cut-out section 38 will be formed near the upper graft adapter section member proximal end 30 on an anterior wall thereof, but, as detailed above, can be varied based on the anatomy of a patient. By depending the location of the upper graft adapter section member third cut-out section 38 on the correspondence with the superior mesenteric artery, the present invention maintains blood flow to the superior mesenteric artery while fixated above the renal arteries 18, 20.

Due to the positioning of the endovascular graft adapter 2 and specifically the upper graft adapter section member 10 above the right and left renal arteries 18, 20, blockage of the superior mesenteric artery can occur by the endovascular graft adapter 2. Thus, the endovascular graft adapter 2 can use portions of the aortic wall above the renal arteries and near the mesenteric artery for stability and fixation by virtue of the formation of the upper graft adapter section member third cut-out section 38 on the upper graft adapter section member 10 while maintaining blood flow to the superior mesenteric artery.

The lower graft adapter section member 12 as shown in FIGS. 1, 6 and 10 further comprises a lower graft adapter section member third cut-out section 40. The lower graft adapter section member third cut-out section 40 is formed in the lower graft adapter section member 12 to correspond with the superior mesenteric artery. In most cases, the lower graft adapter section member third cut-out section 38 will be formed on an anterior wall of the lower graft adapter section member 12 but can be varied based on the anatomy of an individual patient. Furthermore, in most cases, the lower graft adapter section member third cut-out section 40 is formed near the lower graft adapter section member proximal end 34 on the anterior wall thereof, but, as detailed above, can be varied based on the anatomy of a patient. By depending the location of the lower graft adapter section member third cut-out section 40 on the correspondence with the superior mesenteric artery, the present invention maintains blood flow to the superior mesenteric artery while the endovascular graft adapter 2 is fixated above the renal arteries 18, 20.

The upper graft adapter section member third cut-out section 38 extends from the upper graft adapter section member proximal end 30 toward the upper graft adapter section member distal end 32. Similarly, the lower graft adapter section member third cut-out section 40 extends from the lower graft adapter section member proximal end 34 toward the lower graft adapter section member distal end 36. By extending from the respective proximal to distal ends of both the upper graft adapter section member 10 and the lower graft adapter section member 12, the upper graft adapter section member third cut-out section 38 and the lower graft adapter section member third cut-out section 40 maintain proper blood flow to the superior mesenteric artery. As detailed above, the endovascular graft adapter 2 is positioned above the right and left renal arteries 18, 20 while maintaining blood flow not only to the right and left renal arteries 18, 20 but also to the superior mesenteric artery by virtue of the combination of the upper graft adapter section member third cut-out section 38 and the lower graft adapter section member third cut-out section 40.

The orientation of the upper graft adapter section member third cut-out section 38 and the lower graft adapter section member third cut-out section 40, whether more proximal or distal can be varied based on the anatomy of a patient. However, the aforementioned has been described based on placement of the endovascular graft adapter 2 in the anatomy of a normal patient with a normal aorta 8.

Referring to FIGS. 2, 5, and 9, the endovascular graft adapter 2 is shown placed within the aorta 8 with the upper graft adapter section member 10 and the lower graft adapter section member 12 secured each to the other. The upper graft adapter section member 10 and the lower graft adapter section member 12 are secured each to the other in a manner in which the upper graft adapter section member third cut-out section 38 and the lower graft adapter section member third cut-out section 40 are in alignment with each other. This manner of alignment defines an endovascular graft adapter section member third cut-out section that is aligned—with the superior mesenteric artery. Therefore, once the upper graft adapter section member 10 and the lower graft adapter section member 12 are secured each to the other, their respective third cut-out sections 38, 40 are combined to form the endovascular graft adapter third cut-out section, which maintains blood flow into the superior mesenteric artery.

In this manner, the endovascular graft adapter 2 has the ability to be supra renally fixated without inhibiting blood flow to the right and left renal arteries 18, or the superior mesenteric artery due to the formation of the endovascular graft adapter cut-out sections and the endovascular graft adapter third cut-out section respectively.

As shown in FIGS. 1, 6, and 10, the upper graft adapter section member proximal end 30 has a first upper graft adapter section member cross-sectional area and the upper graft adapter section member distal end 32 has a second upper graft adapter section member cross-sectional area. The first upper graft adapter section member cross-sectional area is larger than the second upper graft adapter section member cross-sectional area. In this manner the upper graft adapter section member 10 tapers from a larger cross-sectional area near the proximal end 30 to a smaller cross-sectional area towards a more distal end 32 thereof.

The upper graft adapter section member 10 tapers to a smaller cross-sectional area towards a more distal end thereof to accommodate coupling of the lower graft adapter section member 12.

Referring again to FIGS. 1, 6, and 10, the lower graft adapter section member proximal end 34 has a first lower graft adapter section member cross-sectional area and the lower graft adapter section member distal end 36 has a second lower graft adapter section member cross-sectional area. In this manner, the lower graft adapter section member 12 tapers from a first cross-sectional area closer to a proximal end of the lower graft adapter section member 12 to a smaller second cross-sectional area closer to a distal end thereof. The lower graft adapter section member 12 tapers to a second lower graft adapter section member cross-sectional area that is defined by the secondary stent graft that is attached to the endovascular graft adapter 2. As shown in FIGS. 2, 5, and 9, the secondary stent graft adapter 44 is attached to a distal end of the endovascular graft adapter 2, more specifically to the distal end 36 of the lower graft adapter section member 12. Therefore, the distal end of the lower graft adapter section member 12 will be sized accordingly to be coupled to a secondary stent graft 44. The lower graft adapter section member 12, for example, has a landing zone (a defined area at the distal end 36 on the lower graft adapter section member 12 to couple with and provide an adequate seal with a commercially available stent graft) of approximately 1.5 cm-2 cm and tapers to a diameter of 24 mm. However, the landing zone and the diameter can be varied to couple the endavscular graft adapter 2 to various commercially available stent grafts.

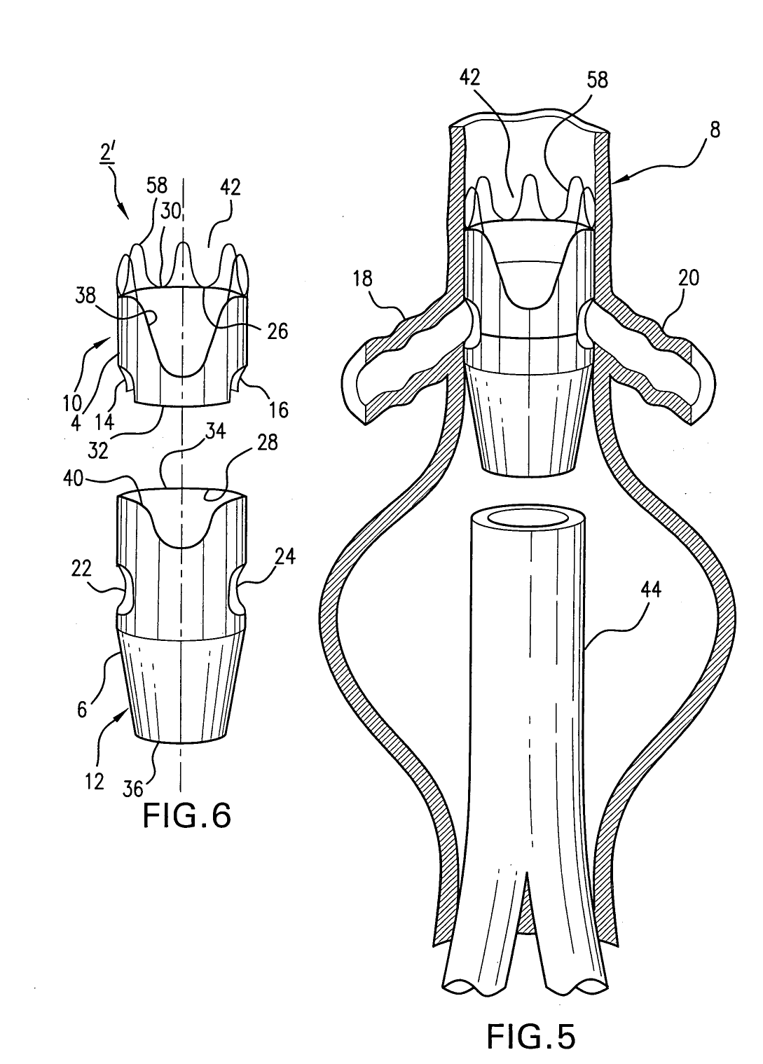

Referring to FIGS. 5-6 and 9-10, there is a section of the endovascular graft adapter 2 specifically the upper graft adapter section member 10 that has a plurality of metallic self-expandable struts forming a stent section 42. The stent section 42 is located above and along an anterior wall of the upper graft adapter section member 10 at the upper graft adapter section member proximal end 30. The metallic self-expandable struts, which expand against the aorta, are disposed extending circumferentially around the upper graft adapter section member proximal end 30 approximately 270°. By extending approximately 270°, the stent section 42 does not occlude the superior mesenteric artery and therefore blood flow is maintained to that artery. In this manner, the stent section 42 will expand against the wall of the aorta thereby enhancing the stability of the endovascular graft adapter 2 within the aorta 8. Furthermore, because the stent section 42 is not covered with any graft material and because it extends circumferentially approximately 270°, it will not occlude blood flow to the superior mesenteric artery or any arteries of the aorta 8. The stent section 42 attached to the endovascular graft adapter 2 allows for a more stable, predictable deployment of the endovascular graft adapter 2 specifically the upper graft adapter section member 10 when inserting within the aorta 8.

Referring to FIGS. 2, 5, and 10, the secondary stent graft 44 is attached to the lower graft adapter section member distal end 36. In this manner, the endovascular graft adapter 2 is formed in a way such that it can be adapted to be used with conventionally known secondary stent grafts 44 based on the anatomy of a patient. Those patients who are not eligible for the use of a secondary stent graft 44 due to poor anatomy, as detailed above, will benefit from the use of the endovascular graft adapter 2, which can be coupled to readily available secondary stent grafts 44 via the distal end 36 of the lower graft adapter section member 12.

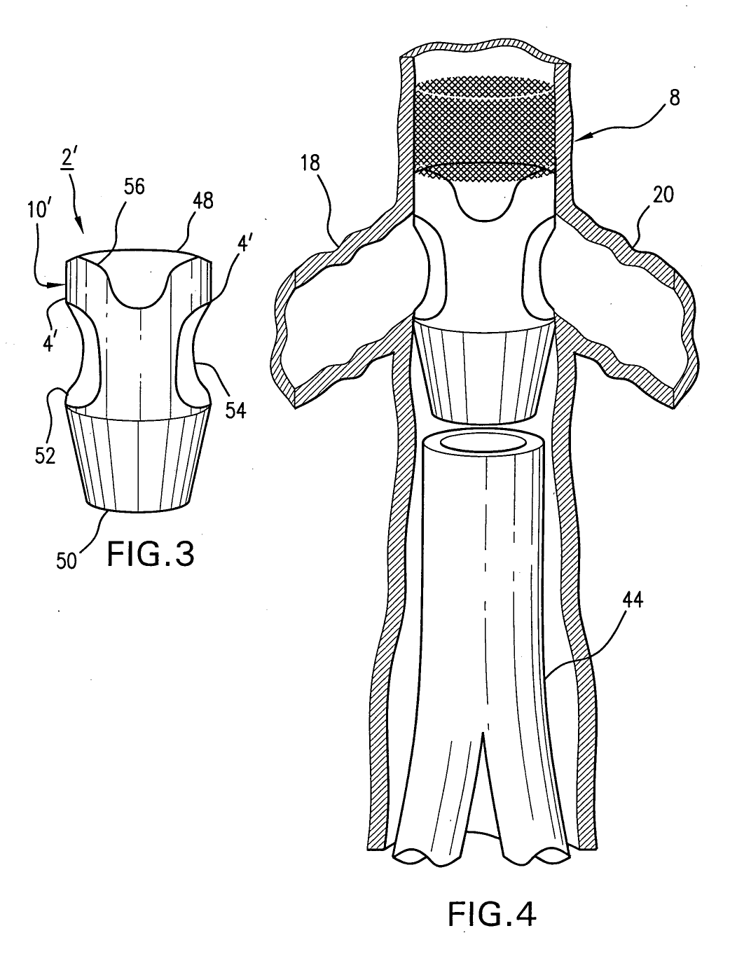

Another embodiment of the endovascular graft adapter 2′ is shown in FIGS. 3-4 and 7-8. The endovascular graft adapter 2′, of this embodiment, is a one-piece structure. There is a graft adapter body 10′ that defines a lumen that extends from a graft adapter body proximal end 48 to a graft adapter body distal end 50. The graft adapter body 10′ has a pair of graft adapter cut-out sections 52, 54. The graft adapter body cut-out sections 52, 54 are formed through a sidewall 4′ of the graft adapter body 10′. When positioned inside the aorta 8 the graft adapter body cut-out sections 52, 54 are respectively aligned with the right and left renal arteries 18, 20. This manner of alignment will not restrict the blood flow through the right and left renal arteries 18, 20 when the graft adapter body 10′ is placed in the aorta 8. Furthermore, the graft adapter body cut-out sections 52, 54 allow for the graft adapter body 10′ to be fixated above the renal arteries in patients that are not appropriate candidates for an endovascular approach, without inhibiting blood flow to the renal arteries 18, 20.

The graft adapter body cut-out sections 52, 54 may be arranged on a sidewall 4′ of the graft adapter body 10′ to accommodate varying orientations of the renal arteries 18, 20 depending on the anatomy of the patient.

Referring to FIGS. 3-4 and 7-8, the graft adapter body 10′ has an axially directed through passage extending from the graft adapter proximal end 48 to the graft adapter body distal end 50. This through passage allows blood flow from the aorta 8 through the graft adapter body 10′ from the proximal end 48 towards the distal end 50 thereof.

The endovascular graft adapter 2′ is self-expanding and thus pushes out circumferentially to seal the blood vessel in which it is placed, i.e. the aorta. The self-expanding nature of the endovascular graft adapter 2′ ensures proper fit within the aorta.

Referring to FIGS. 3-4 and 7-8, the graft adapter body 10′ of the endovascular graft adapter 2′ further comprises a graft adapter body third cut-out section 56. The graft adapter body third cut-out section 56 is formed on the graft adapter body 10′ to correspond with the superior mesenteric artery. In most cases, the graft adapter body third cut-out section 56 will be formed on an anterior wall of the graft adapter body 10′ but can be varied based on the anatomy of the patient. Furthermore, in most cases, the graft adapter body third cut-out section 56 will be formed near the proximal end 48 of the graft adapter body 10′ on the anterior wall thereof, but again can be varied depending on the anatomy of a patient. By depending the location of the graft adapter body third cut-out section 56 on the correspondence with the superior mesenteric artery, the endovascular graft adapter 2′ maintains blood flow to the superior mesenteric artery.

Due to the positioning of the endovascular graft adapter 2′ above the right and left renal arteries 18, 20 blockage of the superior mesenteric artery can occur by the endovascular graft adapter 2′. Thus, the endovascular graft adapter 2′ can use portions of the aortic wall above the renal arteries and near the superior mesenteric arteries for stability and fixation by virtue of the formation of the graft adapter body third cut-out section 56 on the graft adapter body 10′ while maintaining blood flow to the superior mesenteric artery.

Referring to FIGS. 3-4 and 7-8, the graft adapter body third cut-out section 56 extends from the graft adapter body proximal end 48 towards the graft adapter body distal end 50. By extending the graft adapter body third cut-out section 56 from proximal end 48 to distal end 50 of the graft adapter body 10′, the graft adapter body third cut-out section 56 accommodates proper blood flow to the superior mesenteric artery. As detailed above, the endovascular graft adapter 2′ is positioned above the right and left renal arteries 18, 20 while maintaining blood flow not only to the right and left renal arteries 18, 20 but also to the superior mesenteric artery by virtue of the graft adapter body third cut-out section 56.

The graft adapter body 10′ shown in FIGS. 3-4 and 7-8 has a graft adapter body first cross-sectional area at a proximal end 48 thereof. The graft adapter body 10′ has a graft adapter body second cross-sectional area at a distal end 50 thereof. The graft adapter body first cross-sectional area is larger than the graft adapter body second cross-sectional area. In this manner, the graft adapter body 10′ tapers from a larger cross-sectional area near the proximal end 48 to a smaller cross-sectional area towards a more distal end 50 thereof.

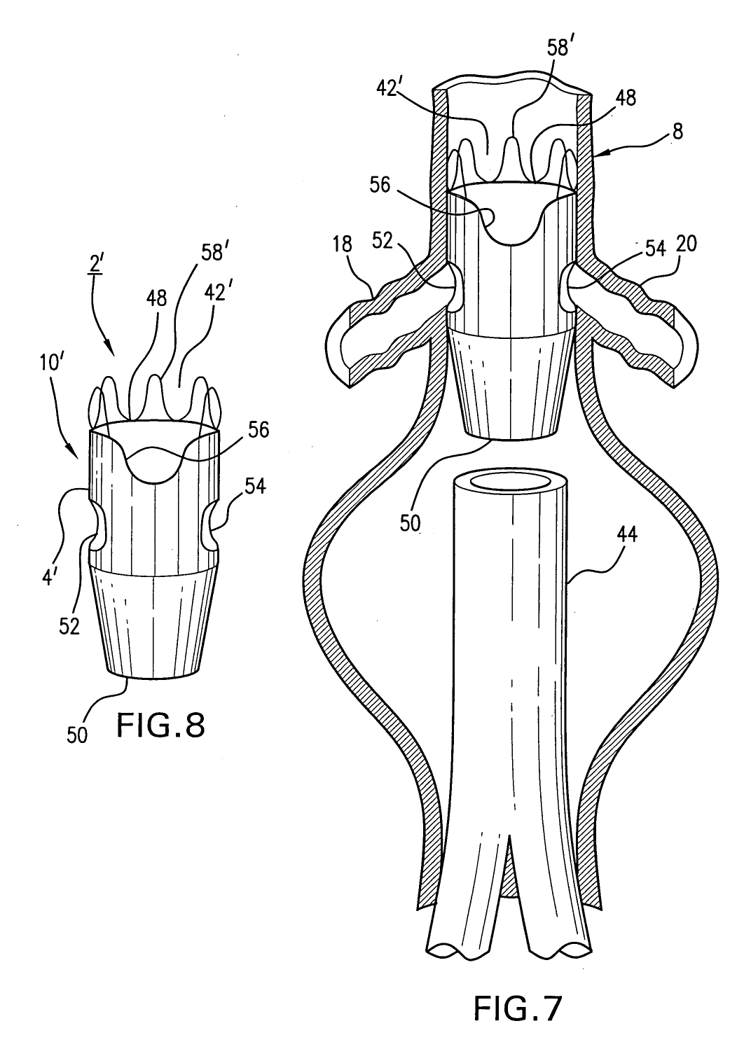

As shown in FIGS. 4 and 7, a secondary stent graft 44 is attached to the distal end 50 of the graft adapter body 10′. Therefore, the distal end 50 of the graft adapter body 10′ will be sized accordingly to be coupled to a conventionally known secondary stent graft 44. The graft adapter body 10′, for example, has a landing zone (a defined area at the distal end 50 on the graft adapter body 10′ to couple with and provide an adequate seal with a commercially available stent graft) of approximately 1.5 cm-2 cm and tapers to a diameter of 24 mm. However, the landing zone and the diameter can be varied to couple the endavscular graft adapter 2′ to various commercially available stent grafts.

Referring to FIGS. 7-8 there is a section of the endovascular graft adapter 2′, specifically the proximal end 48 of the graft adapter body 10′, which has a plurality of metallic self-expandable struts 58′ forming a stent section 42′. The metallic self-expandable struts 58′, which expand against the aorta, are disposed extending circumferentially around the proximal end 48 of the graft adapter body 10′ approximately 270°. Self expanding struts 58′ are formed in an undulating contour as shown in the Figures however a number of conventional contours or shapes may be fabricated which permit expansion against the aorta 8. In this manner, the stent section 42′ expands against the wall of the aorta and enhances stability of the endovascular graft adapter 2′ within the aorta 8 without occluding blood flow to the superior mesenteric artery or any arteries of the aorta 8. Therefore, the stent section 42′ attached to the endovascular graft adapter 2′ allows for a more stable, predictable deployment of the endovascular graft adapter 2′ when inserting within the aorta 8.

Referring to FIGS. 4 and 7, there is shown a secondary stent graft 44 attached to a distal end 50 of the graft adapter body 10′ of the endovascular graft adapter 2′. In this manner, the endovascular graft adapter 2′ is made in a way such that it can be adapted to be used with conventionally known secondary stent grafts 44 based on the anatomy of a patient. Those patient who are not eligible for use of a secondary stent graft 44 due to poor anatomy, as detailed above, will benefit from the use of the endovascular graft adapter 2′, which can be coupled to readily available secondary graft stents 44 via the distal end 50 of the graft adapter body 10′.

The endovascular graft adapter 2, 2′ is composed, generally, of a memory material, such as nitinol and a covering such as polytetrafluoroethylene (PTFE), ETFE (polymer of ethylene and tetrafluoroethylene) or DACRON® (polyester fiber material). The memory metal, such as nitinol is structured to shift between an expanded configuration and a collapsed configuration. Over the metal formed frame, there is a covering of a vascular graft-type material. The covering is structured to prevent leakage, rupture or disconfiguration. The covering, as mentioned, may be polytetrafluoroethylene (PTFE), ETFE or a like polymer material. However, the covering will not cover the stent section 42, 42′, which will remain without any graft-type material, as detailed above.

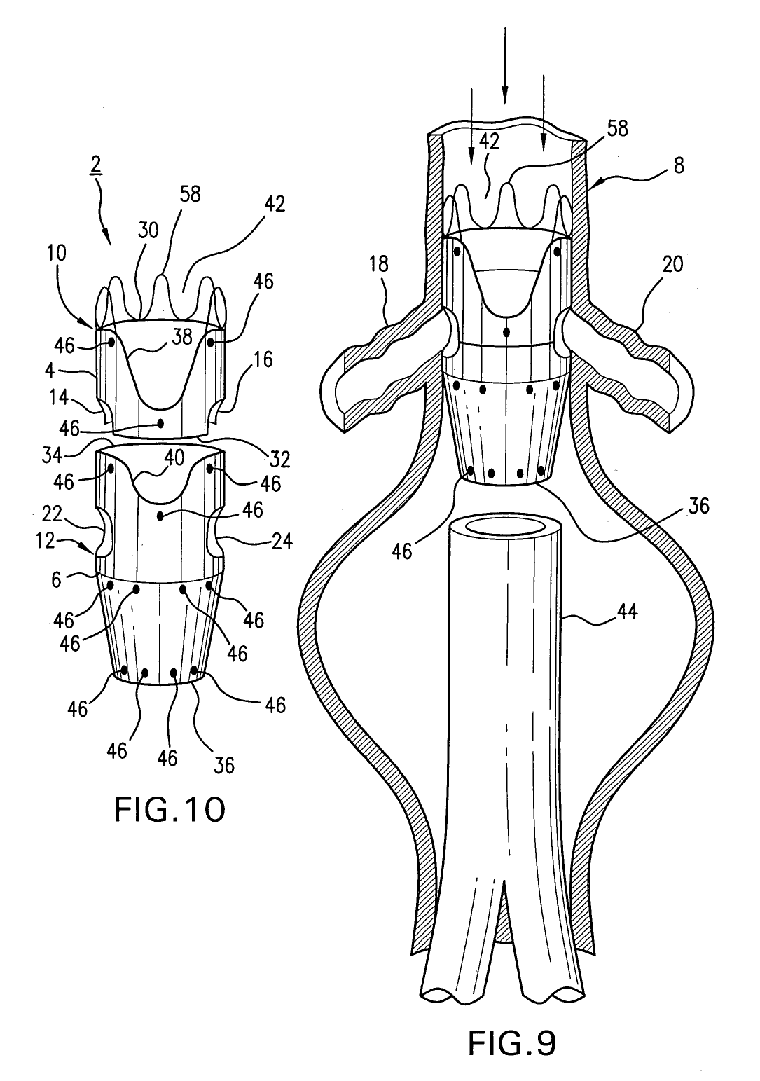

As shown in FIGS. 9-10 to aid in the placement of the endovascular graft adapter 2, there are a plurality of radiopaque markers 46 placed along predetermined points on the upper graft adapter section member 10 and the lower graft adapter section member 12. The radiopaque markers 46 permit a surgeon to easily locate the positioning of the endovascular graft adapter 2 during placement within the aorta 8. Similarly, radiopaque markers 46 can be placed on the one-piece endovascular graft adapter 2′ on various points of the graft adapter body 10′.

It would be appreciated by those skilled in the art that changes could be made to the embodiments described above without departing from the broad inventive concept thereof. It is understood, therefore, that this invention is not limited to the particular embodiments disclosed, but is intended to cover modifications within the spirit and scope of the present invention as defined by the appended claims.

What is claimed is:

1. An endovascular graft adapter for use in abdominal aortic aneurysms comprising:
a graft adapter body defining an upper graft adapter section member and a lower graft adapter section member,
said upper graft adapter section member having a pair of upper graft adapter section member cut-out sections formed through a sidewall of said upper graft adapter section member and respectively aligned with a right renal artery and a left renal artery,
said lower graft adapter section member having a pair of lower graft adapter section member cut-out sections formed through a sidewall of said lower graft adapter section member and respectively aligned with said right and left renal arteries,
said upper graft adapter section member having an upper axially directed through passage extending from a proximal end to a distal end thereof, said lower graft adapter section member having a lower axially directed through passage extending from a proximal end to a distal end thereof, said upper and lower axially directed through passages being in alignment each with respect to the other,
said upper and lower graft adapter section members being secured each to the other at said respective distal and proximal ends thereof.

2. The endovascular graft adapter for use in abdominal aortic aneurysms, as recited in claim 1, wherein said upper graft adapter section member further comprises an upper graft adapter section member third cut-out section formed through an upper graft adapter section member anterior wall at said proximal end thereof and aligned with a superior mesenteric artery.

3. The endovascular graft adapter for use in abdominal aortic aneurysms, as recited in claim 2, wherein said lower graft adapter section member further comprises a lower graft adapter section member third cut-out section formed through a lower graft adapter anterior wall at said proximal end thereof and aligned with said superior mesenteric artery.

4. The endovascular graft adapter for use in abdominal aortic aneurysms, as recited in claim 2, wherein said graft adapter section member third cut-out section extends from said proximal end of said upper graft adapter section member toward said distal end thereof.

5. The endovascular graft adapter for use in abdominal aortic aneurysms, as recited in claim 3, wherein said lower graft adapter section member third cut-out section extends from said proximal end of said lower graft adapter section member toward said distal end thereof.

6. The endovascular graft adapter for use in abdominal aortic aneurysms, as recited in claim 1, wherein said proximal end of said upper graft adapter section member has a first upper graft adapter section member cross-sectional area and said distal end of said upper graft adapter section member has a second upper graft adapter section member cross-sectional area.

7. The endovascular graft adapter for use in abdominal aortic aneurysms, as recited in claim 6, wherein said first upper graft adapter section member cross-sectional area is larger than said second upper graft adapter section member cross-sectional area.

8. The endovascular graft adapter for use in abdominal aortic aneurysms, as recited in claim 1, wherein said proximal end of said lower graft adapter section member has a first lower graft adapter section member cross-sectional area and said distal end of said lower graft adapter section member has a second lower graft adapter section member cross-sectional area.

9. The endovascular graft adapter for use in abdominal aortic aneurysms, as recited in claim 8, wherein said first lower graft adapter section member cross-sectional area is larger than said second lower graft adapter section member cross-sectional area.

10. The endovascular graft adapter for use in abdominal aortic aneurysms, as recited in claim 1, further comprising a stent section, said stent section being located above and along an anterior wall of said upper graft adapter section member at said proximal end thereof.

11. The endovascular graft adapter for use in abdominal aortic aneurysms, as recited in claim 10, wherein said stent section extends circumferentially approximately 270°.

12. The endovascular graft adapter for use in abdominal aortic aneurysms, as recited in claim 1, wherein a secondary stent graft is attached to said distal end of said lower graft adapter section member.

13. The endovascular graft adapter for use in abdominal aortic aneurysms, as recited in claim 3, wherein once said lower graft adapter section member is coupled to said upper graft adapter section member, said upper graft adapter section member third cut-out section is aligned with said lower graft adapter section member third cut-out section and said pair of upper graft adapter section member cut-out sections are aligned with said pair of lower graft adapter section member cut-out sections.

14. The endovascular graft adapter for use in abdominal aortic aneurysms, as recited in claim 1, wherein said endovascular graft adapter is made of polytetrafluoroethylene.

15. The endovascular graft adapter for use in abdominal aortic aneurysms, as recited in claim 10, wherein said stent section is made of nitinol.

16. The endovascular graft adapter for use in abdominal aortic aneurysms, as recited in claim 1, further comprising a plurality of radiopaque markers disposed on said upper graft adapter section member and said lower graft adapter section member.

17. An endovascular graft adapter for use in abdominal aortic aneurysms comprising:
a graft adapter body defining a lumen extending from a proximal end of said graft adapter body to a distal end thereof,
said graft adapter body having a pair of cut-out sections formed through a sidewall of said graft adapter body and respectively aligned with a right renal artery and a left renal artery,
said graft adapter body having an axially directed through passage extending from said proximal end to said distal end thereof.

18. The endovascular graft adapter for use in abdominal aortic aneurysms, as recited in claim 17, wherein said graft adapter body further comprises a third cut-out section formed through an anterior wall at said proximal end of said graft adapter body and aligned with a superior mesenteric artery.

19. The endovascular graft adapter for use in abdominal aortic aneurysms, as recited in claim 18, wherein said third cut-out section extends from said proximal end of said graft adapter body toward said distal end thereof.

20. The endovascular graft adapter for use in abdominal aortic aneurysms, as recited in claim 17, wherein said proximal end of said graft adapter body has a first cross-sectional area and said distal end of said graft adapter body has a second cross-sectional area.

21. The endovascular graft adapter for use in abdominal aortic aneurysms, as recited in claim 20, wherein said first cross-sectional area is larger than said second cross-sectional area.

22. The endovascular graft adapter for use in abdominal aortic aneurysms, as recited in claim 17, further comprising a stent section located on a posterior wall of said graft adapter body at said proximal end thereof.

23. The endovascular graft adapter for use in abdominal aortic aneurysms, as recited in claim 23, wherein said stent section extends circumferentially approximately 270°.

24. The endovascular graft adapter for use in abdominal aortic aneurysms, as recited in claim 17, wherein a secondary stent graft is attached to said distal end of said graft adapter body.

25. The endovascular graft adapter for use in abdominal aortic aneurysms, as recited in claim 17, wherein said endovascular graft adapter is made of polytetrafluoroethylene.

26. The endovascular graft adapter for use in abdominal aortic aneurysms, as recited in claim 23, wherein said stent section is made of nitinol.

27. The endovascular graft adapter for use in abdominal aortic aneurysms, as recited in claim 14, further comprising a plurality of radiopaque markers disposed on said graft adapter body.
